(19) United States
(12) Patent Application Publication
(10) Pub. No.: US 2010/0022910 A1
(43) Pub. Date: Jan. 28, 2010

(54) RAPID ALLERGY TEST

(75) Inventors: John A. Lane, Weedsport, NY (US); Andrew Kugler, Albany, NY (US); Corinn Fahrenkrug, Liverpool, NY (US); Suzanne Abate Gunter, Concord, NC (US); Allan Krauter, Skaneateles, NY (US); Lucas C. McCann, Madison, WI (US); Ashley Elizabeth Tudor, San Francisco, CA (US); Michael D. Smith, San Francisco, CA (US); William Scott, Palo Alto, CA (US)

Correspondence Address: HISCOCK & BARCLAY, LLP, ONE PARK PLACE, 300 S. STATE ST., SYRACUSE, NY, 13202, US

(73) Assignee: Welch Allyn, Inc., Skaneateles Falls, NY (US)

(21) Appl. No.: 12/179,901

(22) Filed: Jul. 25, 2008

(51) Int. Cl. A61B 5/00

(52) U.S. Cl. 600/556

(57) ABSTRACT

A flexible and disposable allergen-containing patch is provided with markings transferable to the skin. The patch may also include piercing elements to become an all-in-one system. A reusable actuator enables a user with little training or experience to administer multiple test injections with the disposable patch. A scope allows magnified viewing of a test site after the skin test is provided, the scope incorporating the patch as a releasable component. An all-in-one applicator assembly that can be bent to fit a convex engagement surface of a rolling actuator is also provided.

### FIELD OF THE INVENTION

The present invention relates generally to skin testing for allergic or other diagnostic reasons. More particularly, the present invention relates to methods and devices related to rapid testing of multiple allergens or multiple levels of allergens that can be administered in the office of a primary care physician or of a specialist.

### BACKGROUND OF THE INVENTION

When confronted with patients having possible allergies, a primary care physician (PCP) has a number of choices. The PCP can refer the patient to a specialist for a relatively involved skin test, often called a scratch test, where, after scratching, abrading, scarifying, or otherwise piercing the epidermal skin layer, suspect allergens are applied to the exposed skin tissue. This form of test can involve multiple visits and involves significant time and cost for the patient. These known tests involve individually filling wells with allergens, which are then applied to the patient by applicators dipped into these wells. Such tests are time consuming, and allow the increased possibility of errors. In addition, this exhaustive, lengthy form of testing isn't always necessary. Alternatively, the PCP can administer blood (RAST) tests, knowing that the results will be delayed for 1-2 weeks and the accuracy is inferior to that of the skin test standard. Often, PCPs recommend their patients to make changes to their environments and/or prescribe medications based solely on patient's medical history. This leads to often confusing results as to the actual cause and some environmental changes (e.g.; removal of a family pet) can be too traumatic to be practical. Therefore, there is a need for a reliable and rapid testing mechanism for on-site allergy testing that a PCP's staff as well as a specialist can administer.

Because there are numerous potential sources for allergic reactions, prior art skin testing apparatuses have been designed in an effort to improve testing time through the use of multi-headed applicators. These applicators, however, tend to be bulky and hard to use, as well as expensive. Moreover, the head of each applicator needs to be loaded with an allergen individually, which is time-consuming and prone to mistakes. Results of such skin testing are often hard to read and cross-contamination of allergens is a general concern that is often not adequately addressed. Therefore, there is a need for simple, economic and reliable testing mechanisms in which multiple allergens can be tested simultaneously on the skin of patients with results that are easily observable.

### SUMMARY OF THE INVENTION

These and other needs are met by rapid allergy testing systems, as provided herein.

According to one aspect, a disposable allergy-testing patch is provided. The patch is defined by a flexible body having a plurality of packets, each said packet housing an allergen, and at least one transferable marking material adjacent each packet that is disposed on one surface of the flexible body. The marking material marks a patient's body upon close contact as an indicator of the allergen applied, a location registration and potentially other information relevant to executing and evaluating the test. In one feature, each packet further houses at least one piercing element that is movably supported within the packet. Retaining means are included for retaining a portion of the allergen when at least a portion of the piercing element is forced through the packet.

In one version, the patch further includes actuation means for moving the piercing element at least partly out of the packet to enable a portion of the piercing element, including the portion that retains allergen from the packet, to extend outwardly from the body. In one embodiment, the actuation means comprises at least one tubular section made from a low durometer elastomeric material that is sized for retaining at least a portion of said piercing element. Each tubular section is configured to deform upon compressive contact of the patch surface with the skin of a patient, permitting the piercing element to move in relation to the tubular portion in order to advance the piercing element in relation to the packet through the tubular portion and into direct contact with the skin.

According to another aspect, an allergy-testing apparatus is provided comprising a disposable patch comprising a flexible body having at least one packet housing an allergen, at least one transferable marking material adjacent said at least one packet and disposed on one surface of said flexible body for marking a patient's skin upon close contact therewith, and at least one piercing element movably contained within said patch and configured to pierce a portion of said at least one packet, said at least one piercing element including retaining means for retaining a portion of said allergen, said at least one piercing element being configured to pierce said packet, wherein said apparatus is configured to advance said retaining means towards said body surface and said at least one transferable marking material into close contact with the skin of a patient indicating the allergen applied and other test-related information.

In yet another aspect, an allergy-testing apparatus is provided having an actuating device comprising a compact body, an actuator mechanism and an engagement surface, said actuator mechanism being operatively connected to at least a portion of said engagement surface such that operation of said actuator mechanism causes spatial displacement of said at least a portion of said engagement surface relative to said compact body, a packet assembly comprising at least one packet housing an allergen, said packet assembly being releasably attached to said engagement surface of said actuating device, and at least one piercing element having a fluid retaining end, wherein spatial displacement of said engagement surface caused by said operation of said actuator mechanism deploys said piercing element such that said fluid retaining end pierces at least one side of said packet, enabling administration of said allergen to a patient.

According to yet another aspect, an allergy-testing apparatus is provided comprising a scope comprising a compact hand-held body, an illumination assembly disposed in connection with said body, an optical system disposed within said body, an engagement surface, and a substrate comprising at least one packet housing an allergen and a piercing element, said substrate being releasably attached to said engagement surface of said scope.

According to still another aspect, a disposable allergen-testing applicator is provided that includes at least one piercing element having an elongated body extending from a base end to a tip end, said tip end comprising at least one sharp tip, said body comprising a shoulder at a predetermined distance relative to said sharp tip, said tip end containing an allergen suspended thereon by at least one capillary action and surface adhesion, at least one enclosure into which said at least one piercing element is disposed, said enclosure having an opening that permits the passage of said tip end, said enclosure being made from a deformable material enabling the tip end of said piercing member to extend beyond said opening upon application of an external force onto said applicator, and a sealing strip covering said enclosures and said piercing elements.

According to one version of the applicator, each enclosure is defined by a substantially Y-shaped wall member disposed around the body of said piercing element, including said tip end, said wall member closely enclosing a portion of said piercing element, from said base end of said piercing element to a deflective point not beyond said shoulder of said piercing element, said wall member extending away from said body of said piercing member between said deflective point and said tip end of said piercing element, said wall member terminating in a rim, and a removable cover sealing said rim of said wall member and forming an interstice around said piercing element's tip end.

According to yet another aspect, an allergen-testing apparatus is provided that includes a body comprising a curved engagement surface, a handle coupled to said body such that operation of said handle causes movement of said curved engagement surface, and a disposable applicator removably attached to and in conforming relationship with said curved engagement surface, said applicator comprising a plurality of piercing elements, each element having an allergen disposed on a tip end thereof, and a corresponding plurality of enclosures retaining said piercing elements, including associated tip ends, each of said enclosures being deformable based on a force applied by a user through said handle to permit the tip end of at least one enclosure to extend, thereby permitting administration of said allergen to the skin of a patient. In one preferred version, the curved engagement surface assumes a convex configuration.

An advantage of the present invention is that the herein described allergy testing apparatuses provide simplified care and provide more timely and cost effective answers during the visit to one's PCP; i.e., quick results to common allergies are available without another appointment or referral to a specialist for lengthy, costly exhaustive testing. Disposable patches containing an array of common allergens or allergen levels can be easily administered by a physician, nurse or medical assistant with minimal discomfort to the patient, simplifying the more extensive and uncomfortable scratch test. Since the injection sites are labeled with temporary marks to facilitate identification of allergic reactions, there is little chance of confusion. Using this invention, the PCP is better able to accurately reach a diagnosis or determine the appropriate next steps (in terms of diagnosis and/or treatment) for those suspected of allergies. In addition to more effectively dealing with specific allergies, a further advantage of this invention is that it reduces the expense and risk of overtreatment of patients who do not actually have allergies, or do not have a suspected allergy.

Still other aspects, embodiments and advantages are discussed in detail below with regard to the following Detailed Description, which should be read in conjunction with the accompanying drawings.

### BRIEF DESCRIPTION OF THE DRAWINGS

FIG. 1A is a perspective view of an embodiment of an allergen-containing patch;

FIG. 1B is different perspective view of the patch illustrated in FIG. 1A;

FIG. 1C is a cross-sectional view of two alternative embodiments of allergen-containing packets that can be found in the patch illustrated in FIGS. 1A and 1B;

FIG. 2A is a perspective view of an actuator made according to an embodiment wherein the actuator is shown in an inactive state with a patch illustrated in FIGS. 1A and 1B about to be attached thereto;

FIG. 2B is a perspective view of the actuator illustrated in FIG. 2A in an activated state;

FIG. 2C is a perspective view of the actuator illustrated in FIGS. 2A and 2B loaded with the patch illustrated in FIGS. 1A and 1B,

FIG. 3 is a perspective view of two alternative embodiments of a piercing element;

FIG. 4 is a cross-sectional view of an allergen-containing packet in an all-in-one disposable patch as provided according to one embodiment;

FIG. 5 is a perspective view of an actuator with the all-in-one disposable patch illustrated in FIG. 4 ready to be attached thereon;

FIG. 6 is a cross-sectional view of the packet of FIG. 4 as acted upon by the actuator illustrated in FIG. 5;

FIG. 7 is a perspective view of a scope according to an embodiment;

FIGS. 8A-8C are perspective views of a user using the scope illustrated in FIG. 7.

FIG. 9 is a perspective view of an all-in-one applicator assembly in accordance with an embodiment;

FIG. 10A is a cross-sectional view of an individual applicator embodiment;

FIG. 10B is a cross-section view of the individual applicator of FIG. 10A as it is being applied to inject into the patient skin;

FIG. 11 is a perspective view of an actuator loaded with the applicator assembly of FIG. 9;

FIG. 12 is a perspective view of a user using the loaded actuator of FIG. 11; and

FIG. 13 is a perspective view of a user using the loaded actuator of FIG. 11 in an alternative way.

### DETAILED DESCRIPTION

The invention is herein described, by way of example only, with reference to the accompanying drawings. It is stressed that the particulars shown in the drawings are by way of example and for purposes of illustrative discussion of the preferred embodiments of the present invention only, and are presented to provide exemplary versions to provide a proper understanding of the advantageous features of the allergy test apparatus. Other versions should be readily apparent to those of ordinary skill embodying the concepts described herein. In addition, certain terms are used throughout in order to provide a suitable frame of reference with regard to the accompanying drawings. These terms, however, are not intended to be limiting, except where specifically noted.

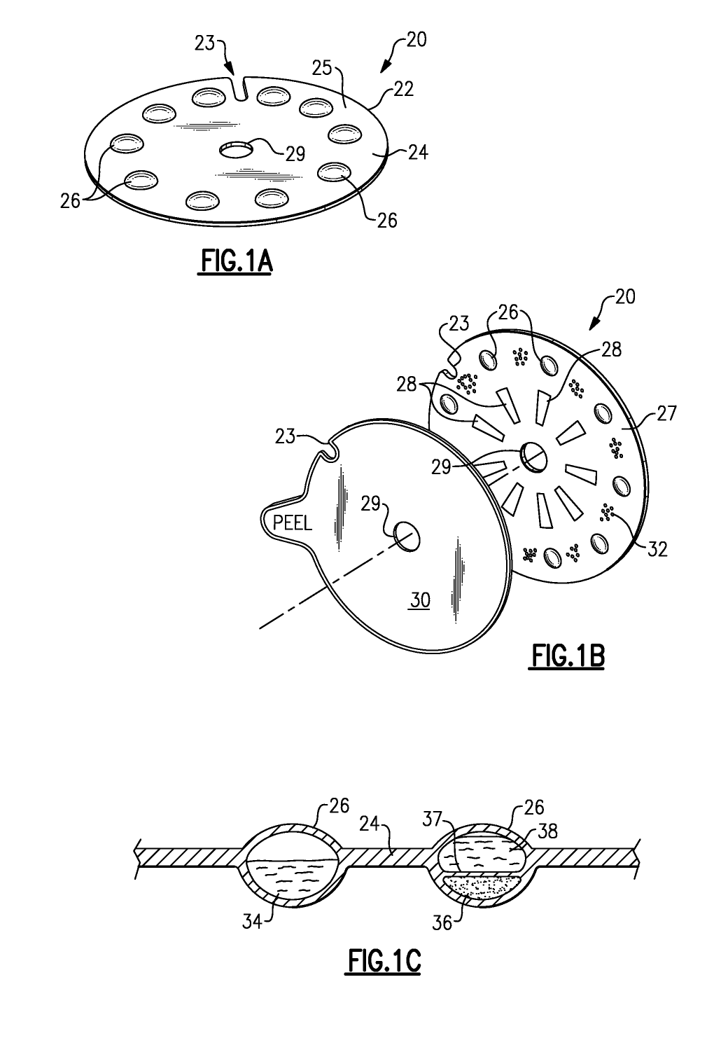

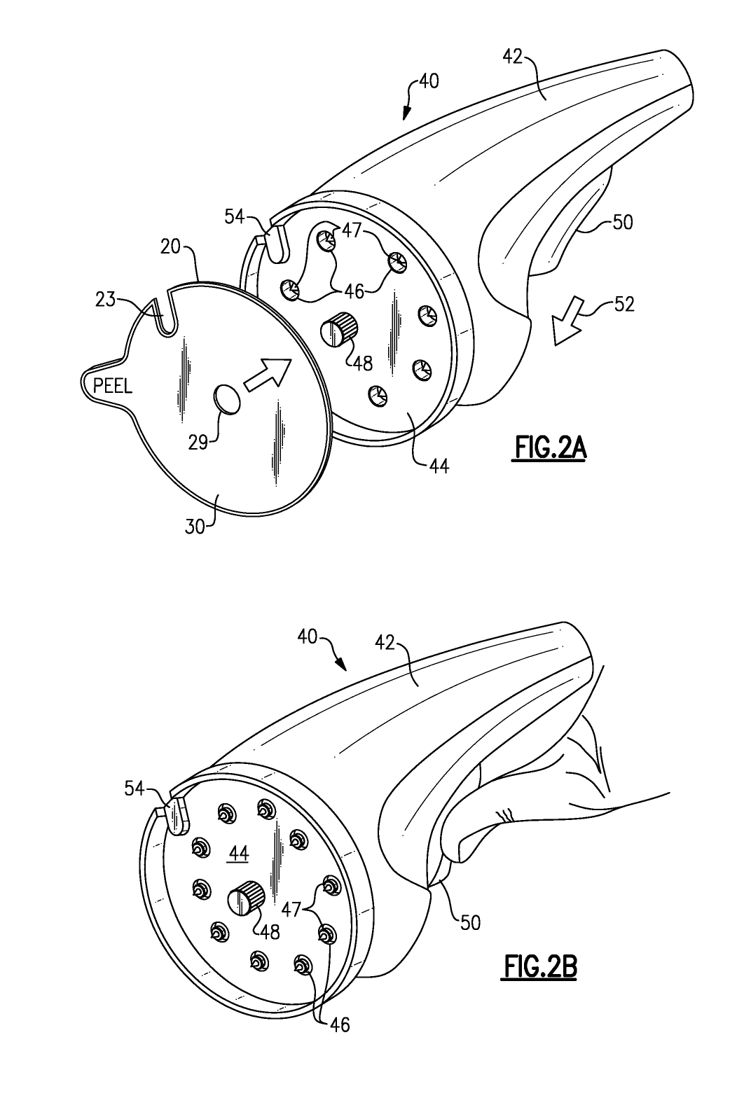

Referring to FIGS. 1A-1C, a first embodiment of a rapid allergy testing system is shown and described herein. A disposable patch 20 is defined by a body 22 that can be applied directly onto the skin of a patient for an allergic skin test. The patch body 22 consists of a relatively planar substrate 24 having a plurality of small blisters or packets 26 thereon. The patch body 22 may further include at least one position marker, e.g., a notch 23 and a central bore 29, that help to position and load the patch 20 onto an actuator or applicator, which will be explained in further detail later. In one embodiment, the substrate 24 is flexible to accommodate the natural contours of the area of the skin of the patient that the apparatus is to be applied on. However, the substrate 24 can be relatively stiff as well.

The packets 26 may protrude from the patch's top surface 25 or the bottom surface 27 or both surfaces. The packets 26 may be made from the same material as the substrate 24 or from a different though preferably flexible and fluid-impermeable material. Sealed within each of the packets 26 is either an allergen or a control reagent. In one embodiment, each packet 26 also contains at least one piercing element (not shown). Obviously, the number and distribution pattern of the packets 26 may vary as well as the size and shape of the patch 20. In the embodiment shown, the disposable allergy-testing patch 20 is substantially round, about 1-2 inches in diameter, and has about ten small packets distributed about its perimeter. In one embodiment, each of the packets 26 contains one of the ten most common allergens that cause, in combination, a majority, e.g., about 90%, of the environmental allergies, including, for example, pollens, dust mites, mold spores, latex, insect venom, medicines, etc. In another embodiment, each of the packets 26 contains one of the ten most common allergens that cause, in combination, a majority, e.g., about 90%, of the food allergies, including, for example, milk, eggs, peanuts, tree nuts, fish, shellfish, soy, wheat, etc. In yet another embodiment, the packets 26 represent a subcategory of the environmental or food allergies, e.g., grass, and will represent, say, ten different types of common grasses. Optionally, one or two of the packets 26 can contain a positive control and/or a negative control, respectively.

Specifically referring to FIG. 1B and on the bottom surface 27 of the patch 20, adjacent at least one and preferably each packet 26 is a marking material 28 that is transferable to the patient skin. The marking material 28 can be used to indicate the identity of the allergen or control reagent contained in each packet 26. The marking material 28 may directly spell out the packet content, (e.g., ragweed, cat or peanut). Alternately, the marking material 28 may be in the form of alphanumerical symbols that, when cross-referenced to a chart (not shown), informs the user of the identity of the packet's content. The marking material 28 may further include other information relevant to executing and evaluating the allergy test, e.g., a serial number for the particular test administered. The marking material 28 may be an ink, paint, or comprised of other materials that transfer onto the skin upon close contact. The marking material 28 may be water-proof or sweat-proof to ensure legibility after the predetermined time for the skin to be exposed to the allergen has passed. The marking material 28 should be temporary in nature. A removable cover 30 is optionally included on the bottom surface 27 of the patch 20 such that the marking material 28 is protected and that the bottom surface 27 remains sterile before use. The removable cover 30 or the top surface 25 of the patch 20 may also mark the allergen contents for visual confirmation. The patch's bottom surface 27 may also be coated with an adhesive 32 that, once exposed, facilitates extended adhesion to patient skin.

An advantage of having at least one marking material 28 on the testing patch 20 that transfers directly unto the skin is that it allows simultaneous testing of multiple patches on the same patient without having to worry about mixing up test results, and hence saving time for both the healthcare staff and the patient. This is particularly advantageous in the case of testing children on whom, due to the limited size of testable area, more patches could be needed for testing the same number of allergens than on adults.

An advantage of marking all of the test sites is that it allows the practitioner to rapidly and accurately identify the allergen responsible for an allergic reaction. It also prevents possible errors made using the current technique where allergens are poured into wells that the injectors are then dipped into before application. The marking further serves as a reference as to the location of the overall patch as applied.

Specifically referring to FIG. 1C, two of the packets 26 are shown in cross-section. In the example depicted on the left, the packet 26 contains a quantity of liquid allergen, typically, an allergen extract suspended in a solvent. The example on the right illustrates a solution in which the allergen in suspension does not have adequate shelf life for intended use. Therefore, a separation barrier 37 is built between the freeze-dried allergen 36 and the solvent 38 (e.g., saline). In operation, the user performs a motion to puncture the separation barrier 37, and optionally, shakes to mix the contents before applying to the patient skin.

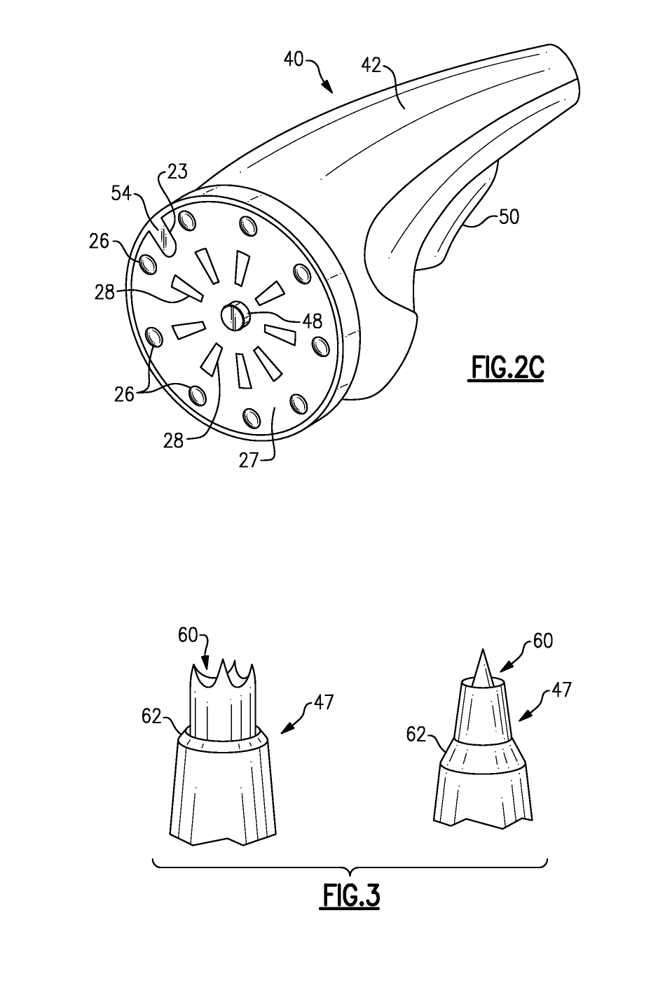

Referring now to FIGS. 2A-2C, an applicator or actuator 40 is provided for use with the disposable patch 20. The actuator 40 is defined by a handle or a compact body 42 having a distal engagement surface 44. Channels 46 are provided on the engagement surface 44 that match the packets 26 on the disposable patch 20 in terms of both number and distribution pattern. Inside each of the channels 46 is a piercing element 47. FIG. 2A shows the actuator 40 in a default or inactive state in which each piercing element 47 is disposed inside the channel 46 behind the engagement surface 44, and only its tip is barely visible. Each piercing element 47 is operatively linked to an actuation button 50 through a spring-loaded mechanism. The spring (not shown) is biased towards the inactive state, such that, to active the piercing elements, the user has to push the actuation button 50 in the direction shown by arrow 52 to counter the spring force. Upon release of the actuation button 50, the spring will force the piercing elements 47 back into their respective channels 46. Such a mechanism and its alternatives for achieving a similar result are well known and the details are omitted here. FIG. 2B shows the actuator 40 in an activated state, where the user could keep pressure on the actuation button 50 in order to keep the piercing elements 47 extended outside their respective channels 46. The piercing elements 47 will only extend as far as the mechanism allows, so there will be much more assurance in the uniformity of piercing depth no matter how much experience or training the user has. Further, because all the piercing elements 47 are connected to the same actuation mechanism, with one actuation operation, the operator can achieve the same or similar depth of all the linked piercing elements 47, thereby injecting similar levels of various allergens to achieve valid comparisons to each other and to the controls. Because penetration depth is fixed and predictable, cross-contamination that occurs with excessive allergen can be avoided.

A central pin 48 matches the central bore 29 of the disposable patch 20, so that a user can align the two to load the patch onto the engagement surface 44. A protrusion 54 is provided on the engagement surface 44 that matches the position notch 23 on the patch 20 such that each packet 26 aligns with a piercing element 47. After the patch 20 has been loaded onto the engagement surface 44, the cover 30 is removed from the patch to reveal each packet 26 along with the adjacent transferable marking material 28 on the patch bottom surface 27 (FIG. 2C). The user then presses the loaded engagement surface/patch bottom surface onto a patient's skin. Suitable testing areas include certain traditional areas, such as the forearms and the back, which provide relatively flat surfaces. The engagement surface 44 of the actuator 40 may be made of a flexible material or can optionally be padded to take advantage of any flexibility in the disposable patch 20. In that case, the testable skin area broadens to areas on the body previously considered as not being insufficiently flat. This broadening reduces testing time as more patches can be tested on the same patient at the same time, and could reduce office visits by the same patient who otherwise may have run out of skin area for testing during one visit.

Once the patch 20 has made full contact against the patient body, the user activates the actuator 40 by simply pressing on the button 50. Each hidden piercing element 47 pierces through both surfaces of each packet 26 into the skin, resulting in rapid injection of test reagents. Rapid injection of multiple allergen tests is advantageous because it minimizes discomfort for the patient. Piercing elements useful for skin scratch tests are well known in the art, and typically have fluid retaining ends that, through capillary action, retain sufficient amounts of allergen extracts to result in visible allergic reactions. Some examples of piercing elements 47 are shown in FIG. 3, with fluid retaining ends 60 and shoulders 62 to limit the element's penetration depth into the skin. U.S. Pat. Nos. 4,966,159, 5,820,562, 5,931,794 and 6,024,706 commonly disclose piercing elements that can be used in the present invention and their entire disclosures are hereby incorporated by reference. Shoulder stops 62 give further assurance of penetration depth control, the benefits of which have been described above.

After the user administers the skin scratch test using the actuator 40 and patch 20 of the present apparatus, the actuator loaded with the patch 20 is removed from the patient body. The patch 20 is peeled off from the actuator 40 and discarded. Transferable marking material 28 has automatically labeled each injection site, so that when the user inspects afterwards, there is no confusion as to which allergen has caused a reaction. Typically, the user waits for about 15 minutes before looking for positive reactions such as wheals above a certain size (e.g., about 4 mm in diameter) on the patient's skin.

Accordingly, the rapid allergen testing systems described herein are easy to use and provide substantial information in a short period of time, with accuracy comparable to that of existing scratch tests and higher than that of blood/RAST tests. Armed with this information about potential allergies, a PCP can make a more informed decision on succeeding steps (e.g., other testing for source of symptoms, treatments, referral to a specialist, etc.).

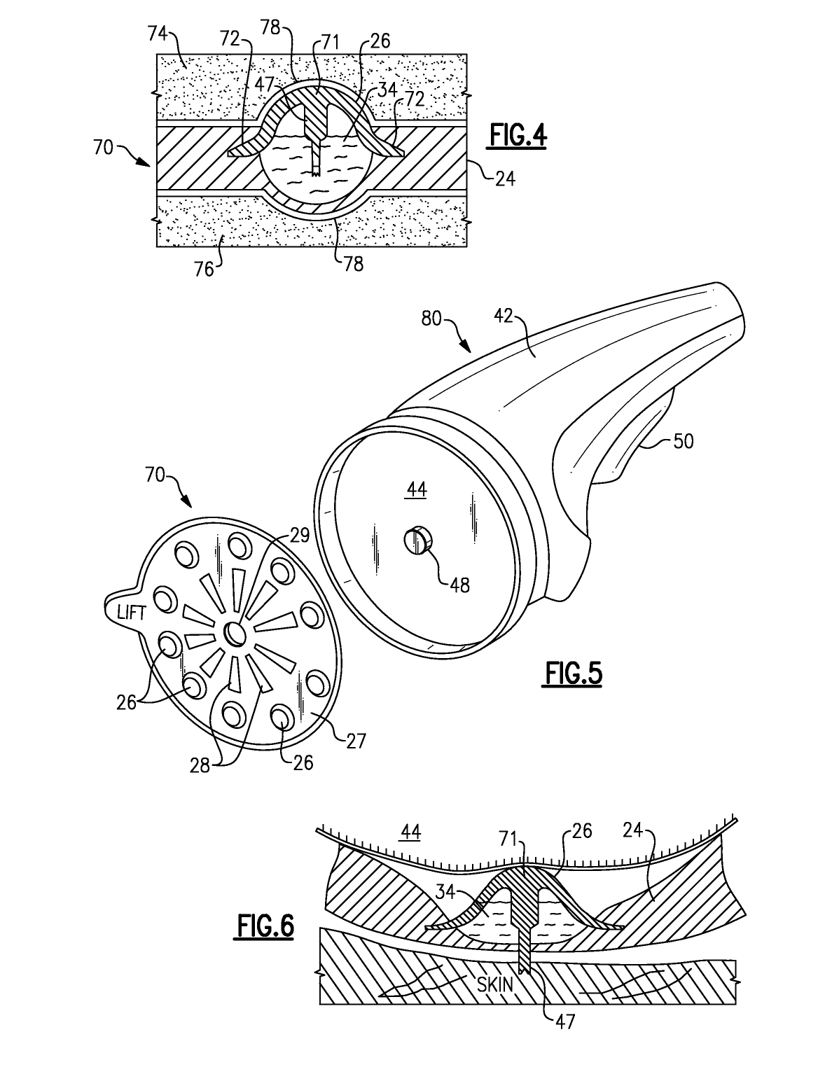

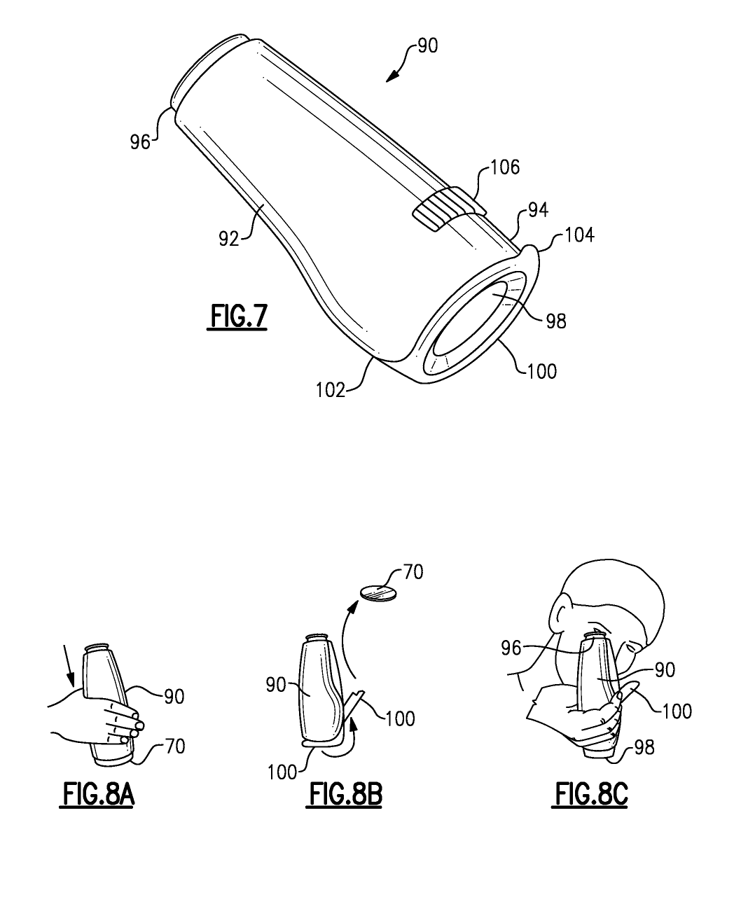

Referring now to FIG. 4, an all-in-one embodiment of the disposable patch 70 is shown in cross-section. Parts similar to those of previous embodiments are labeled with the same reference numerals and their properties, unless noted, are similar and not repeated here. The packet 26 shown in FIG. 4 has at least one piercing element 47 with a support 71. In the example shown, the support 71 is substantially dome-shaped and integral with the piercing element 47. Of course, the support 71 can be of other configurations and shapes, and/or can be a separate part from the piercing element 47. The support 71 may have a rim 72 around it that extends into the patch substrate layer 24—the rim 72 provides surface area to sealingly bond the support 71 to the substrate 24. In one feature, the substrate is made from a relatively flexible material, e.g., soft plastic or rubber elastomer, while the support 71 is made from a material with higher durometer/hardness, e.g., hard plastic. This preserves the overall flexibility of the patch 70 while providing necessary support for the piercing element. For storage, in one embodiment, the all-in-one patch 70 is immobilized in between two racks 74 and 76, both made of hard plastics and molded with matching cutouts 78 for accommodating the packets 26. As shown in FIG. 5, similar to patch embodiments described earlier, the all-in-one patch 70 may also have at least one transferable marking material 28 on the bottom surface 27 for automatically labeling the allergen packets during close contact with patient skin. The all-in-one patch 70 does not need to have a position notch on its perimeter, as the description below makes this evident. Also, if the storage racks 74 and 76 can form an airtight seal or are packaged in one, there is no more need for a removable cover on the patch 70 itself.

The all-in-one patch 70 described herein can be manually applied, i.e., a user can administer the scratch test by applying pressure directly by his or her hand on the outside of the support 71 to pierce each packet 26 and to “scratch” the skin. However, to rapidly inject multiple test allergens with uniform pressure, according to one feature of the invention, an actuator 80 is provided for applying and actuating the all-in-one patch 70. Referring to FIG. 5, the actuator 80 is similar to actuator 40 except it does not provide any piercing element and no protrusion is needed on the engagement surface 44. The spring-loaded actuation mechanism connects the engagement surface 44 with the actuation button 50. When the actuation button 50 is pressed, the engagement surface 44 is displaced in a distal direction relative to the stationary central pin 48 and the compact body 42. Similar to using the actuator 40 described previously, with the actuator 80, the operator can achieve the same or similar depth for all the piercing elements 47, thereby injecting similar levels of various allergens to achieve valid comparisons to each other and to the controls while avoiding cross-contamination from allergen overrun. Alternately, actuator 80 could have the form of an annular ring or other suitable forms.

When administering the test, the all-in-one patch 70 is first loaded onto the actuator 80 by matching the patch's central bore 29 onto the central pin 48, and with the top surface, the side that has the relatively hard support 71 for the piercing elements, facing the engagement surface 44. Then the user contacts the bottom surface 27 of the patch 70, as much as possible, with a skin testing area of the patient. Subsequently, the user presses the button 50 on the actuator 80 to push the engagement surface 44 towards the patient. As shown in FIG. 6, this actuation forces the flexible patch substrate 24 to flex where it meets the relatively inflexible support 71 for the piercing element 47, effectively forcing the piercing element 47 through one side of the packet 26 and into the skin where it applies an amount of allergen 34 stored in the packet 26. The remainder of the testing procedure is similar to the one described previously, with the all-in-one patch 70 discarded and the actuator 80 ready to be reused with a new patch.

Referring now to FIG. 7, a scope with lighting to aid the reading of possible skin reaction is provided according to one exemplary aspect. In this example, a scope 90 is defined by a compact, hand-held body 92. An illumination assembly (not shown), e.g., a battery-powered light-emitting diode (LED) or conventional incandescent bulb or lamp, is disposed in connection with the body 92, e.g., inside the bore of the body 92, so that when enabled, illumination is provided in the field of view in front of the distal end 94 of the scope 90. In one embodiment, a light switch 106 is provided on the exterior of one side of the scope body 92. An optical system disposed within the scope body 92 includes at least the eyepiece 96 as well as a magnifying lens 98 situated at the scope distal end 94. The eyepiece 96 and the lens 98 are aligned along an optical axis extending through an engagement surface 100.

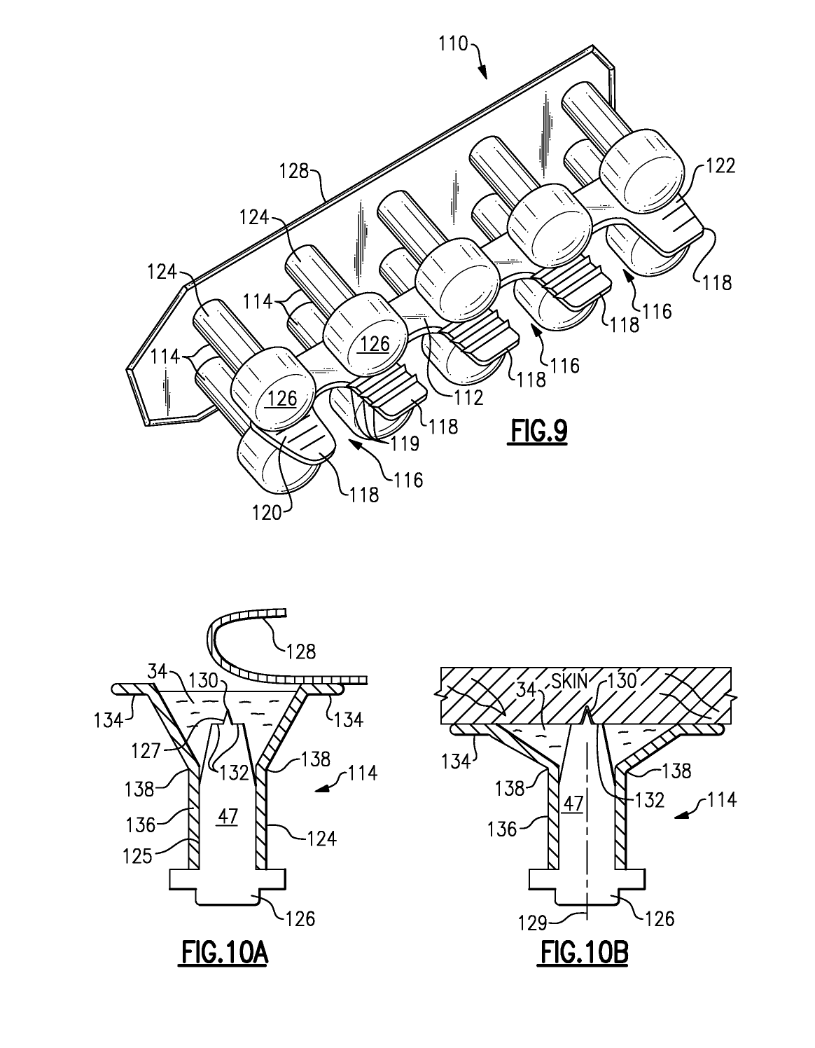

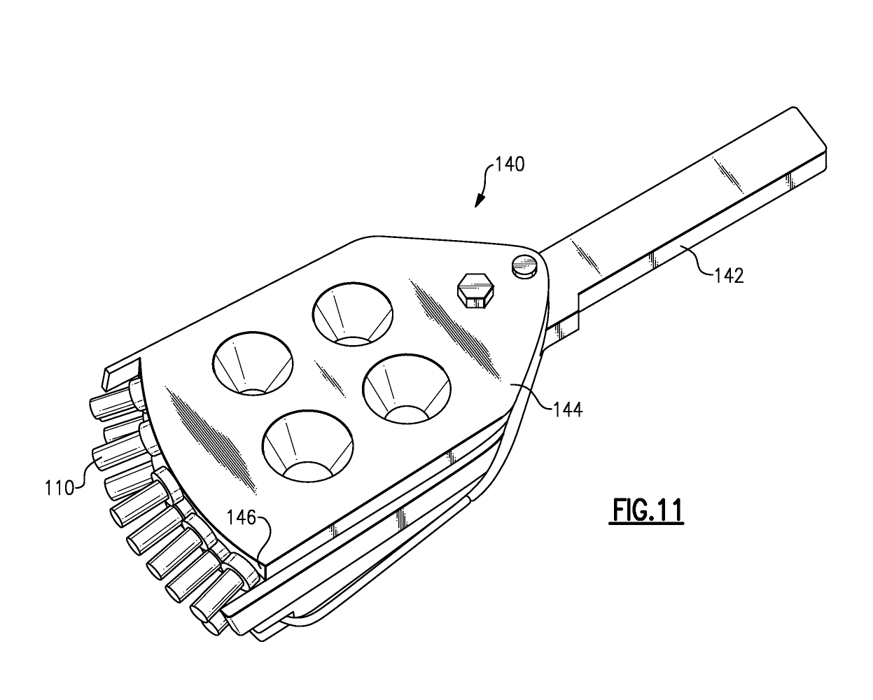

The engagement surface 100 is provided to serve a similar function as the engagement surfaces described above in association with other actuator embodiments of the present invention, and can be used in those embodiments as well. Benefits described with regard to other actuator embodiments, such as uniform depth control, apply here as well. In this specific example, the engagement surface 100 is formed in a ring configuration, which is sized and shaped to fit over the scope distal end 94 and is also connected to one side of the scope body 92 through a hinge 102. As a result, a user can lift a tab 104 opposite the hinge 102 and ply the engagement surface 100 from the scope distal end 94, enabling the surface to be flipped or lifted hingeably to an opposing side (FIG. 8B). This lifting action clears the view for the lens 98 and the remainder of the optical system for the user to look through the scope 90 in an unobstructed manner. Of course, there may be many different forms for the engagement surface, e.g., a full circular cap, and the invention is not limited to the particular form illustrated here. In one feature, the allergen-containing patches of the present invention can be releasably attached to the engagement surface 100. For example, a temporary adhesive or a frictional fit may be used for that purpose.

Referring now to FIGS. 8A-8C, operation of the scope/actuator 90 is now described. After attaching an all-in-one patch 70 to the engagement surface 100, and making sure that the ring of the engagement surface 100 is in substantial contact with all the packets 26 of the patch, the loaded scope 90 is pressed against a patient's skin and pressure is applied to the skin in a stamping motion and/or a subsequent, controlled grinding or circular motion in order to apply the allergen (FIG. 8A). Once all the injections have been administered, as described above, the scope 90 is lifted from the patient and the all-in-one patch 70 is removed from the engagement surface 100 and discarded. When it is time to inspect the skin test site, the user flips up the engagement surface 100 (FIG. 8B) and switches on the illumination assembly in the scope body so that the practitioner can look through the eyepiece 96 and the lens 98 at the test site (FIG. 8C).

Optional elements of this embodiment include a measuring reticle integral to the optical system. Using this reticle, the user may more quickly determine if the criteria for a positive reaction has been met (e.g., wheal of over 4 mm diameter, etc.).

Because the scope 90 is able to illuminate and magnify the view at the test site, diagnostic accuracy is improved. Further, smaller piercing elements and less amount of allergen may be extract needed, reducing the footprint of each individual allergy test. Accordingly, more allergy tests can be fitted into the same amount of skin area or the same number of allergy tests requires less skin area. This not only increases the efficiency of allergy testing, but also reduces discomfort for the patient, and is especially advantageous in pediatric care.

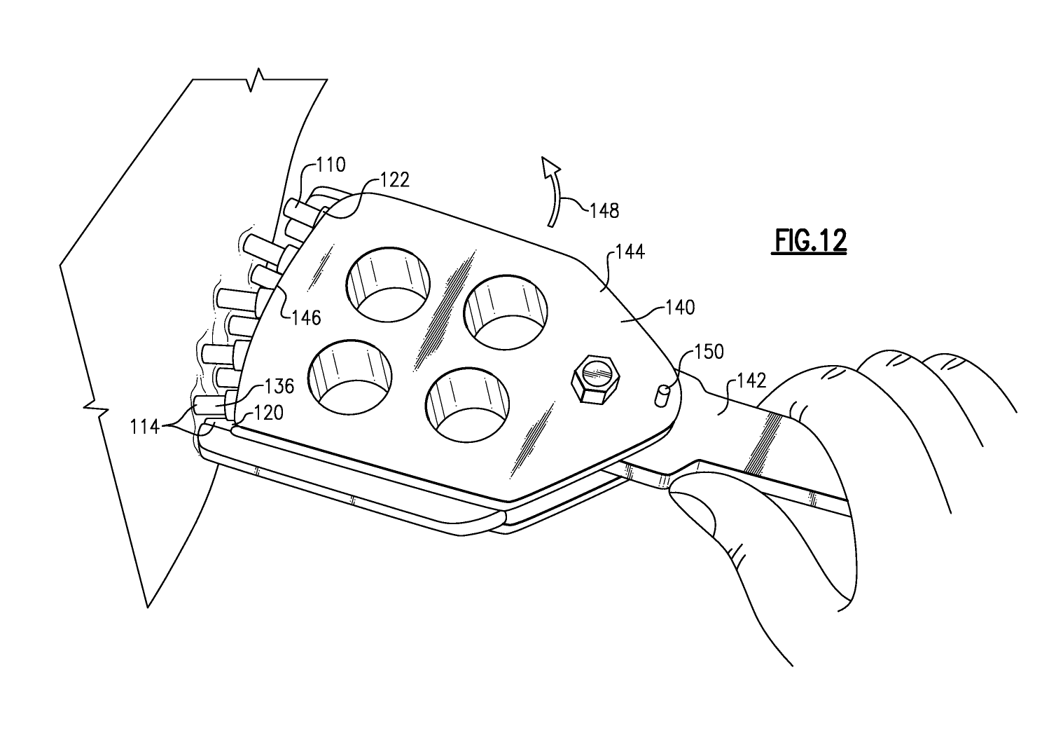

Referring now to FIG. 9, an all-in-one applicator assembly 110 that can be used to rapidly administer multiple allergen injections is herein described according to another aspect. The applicator assembly 110 includes a bridge 112 that serves as the backbone of the assembly, connecting multiple applicators 114 (in the illustrated example, ten assembled in two rows), and also provides a structure for handling. Moreover, and according to this exemplary embodiment, the bridge 112 has four cutouts 116 and five corresponding extensions 118 (with multiple optional ridges 119 thereon) for assembling onto an actuator, which will be described further below. Having cutouts 116 spaced out along the primary or long axis 129, FIG. 10B, of the applicator assembly 110 also increases flexibility of the bridge 112, which may be made of any suitable material, (e.g., a polypropylene) having suitable elasticity. This elasticity allows the user to grab the two ends 120 and 122 on the long axis and bend the assembly 110 off its long axis. This is advantageous for assembling onto a curved engagement surface, which is ultimately advantageous for applying to a curved body surface, which will be described further below.

Each individual applicator 114 houses one of an allergen extract or a control reagent and at least one piercing element (not shown). Each individual applicator 114 includes a vial 124 and a base 126. In the illustrated example, the vial 124 has a substantially uniform diameter—as will be described below, that is not the only configuration useful for the present invention. A removable sealing strip 128 caps the distal tips of all the applicators 114 and retains the allergen extract inside the applicators 114 until use. In one version, the entire applicator assembly 110 can be manufactured from disposable materials and discarded after a single use or single-patient use.

Referring now to FIGS. 10A and 10B, detailed views of a preferred embodiment of an individual applicator 114 are provided in cross-section. Inside the vial 124 is a piercing element 47 defined by an elongated body 125 extending from a base 126 to a tip end 127 that has at least one sharp piercing tip 130. The piercing tip 130 retains some allergen fluid through capillary action and surface adhesion once the piercing element 47 is extended outside the vial 124. Moreover, the piercing tip 130 extends from a shoulder 132 that serves as a stop against further advance into the skin. As the shoulder 132 is set at a predetermined distance from the piercing tip 130, the maximal penetration depth of the piercing element 47 is predetermined and therefore is well controlled. Other configurations for the piercing tip 130 and the shoulder 132, including those described above in association with other embodiments, can also be used here.

The vial 124 provides enclosure for a reservoir around the piercing tip 130 that stores a liquid allergen extract 34 or a control reagent (positive or negative). The vial's distal rim 134 defines a distal opening that is covered by the removable seal 128. In one feature, the vial wall is made from a deformable material that collapses, folds or flexes to allow the piercing tip 130 to extend beyond the distal opening when an external force tries to move the applicator 114 in the distal direction against a barrier, such as the skin. In a preferred embodiment, the vial wall consists of a substantially Y-shaped wall member 136 around the piercing element's elongated body 125, i.e., the wall member 136 closely encloses the portion of the elongated piercing body 125 from the base 126 to a flex point 138 that is below the shoulder 132 of the piercing element 47. The vial wall member 136 extends away from the piercing element's elongated body 125 between the flex point 138 and the piercing tip 130, terminating at the distal rim 134. As noted above, the sealing strip 128 seals the rim 134. However, this strip 128 also forms an interstice around the piercing tip 130 that is normally filled with a liquid allergy extract or control reagent. In one embodiment, the vial wall member 136 is molded from a relatively low durometer elastomer, e.g., silicone rubber and the like.

Referring specifically to FIG. 10B, in administering the skin test, the user first peels off the sealing strip 128, and then contacts the patient's skin with the distal rim 134 of the applicator 114. As the user continues to apply an axial force in the distal direction against the patient's skin, the vial wall member 136 begins to bend away from the long axis 129 of the applicator 114, especially if the vial wall member is made of a low durometer material. Eventually, the vial wall member 136 starts to flex at the flex point 138, allowing the piercing tip 130, along with allergy extract retained thereon, to extend distally past the distal vial rim 134 and into the skin. Penetration of the skin stops at the shoulder 132 of the piercing element 47. As the distal vial rim 134 is pushed against the skin, it also seals the allergen fluid locally around the puncture wound, preventing cross contamination of allergens.

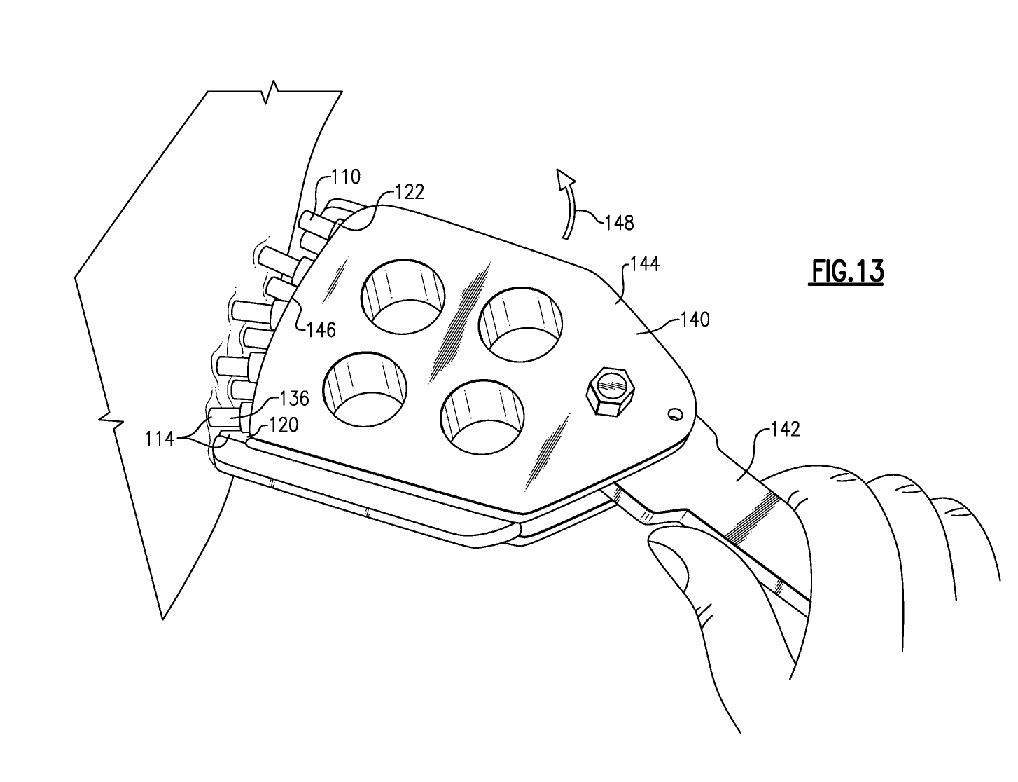

Referring now to FIG. 11, an actuator 140 is provided for applying the applicator assembly 110. In one embodiment, the actuator 140 has a handle 142 coupled to a body portion 144, the distal extremity of which is an engagement surface 146. In this exemplary version, the engagement surface 146 is curved, in a convex configuration (though this configuration could also be concave, especially for applications over the limb). The engagement surface 146 also defines a groove (not shown) that, in cooperation with the bridge 112 (FIG. 9) of the applicator assembly 110, allows the applicator assembly to be installed there onto. In one embodiment, the engagement surface 146 also has depressions (not shown) that correspond to the extensions 118 (FIG. 9) of the applicator assembly 110. As described above, with cutouts, the bridge of the applicator assembly 110 is flexible enough that, after removal of the sealing strip 128, the user can bend the assembly 110 to be installed in that bent shape onto the engagement surface 146 of the actuator 140.

Referring now to FIGS. 12 and 13, use of the actuator 140 and the applicator assembly 110 is hereby described. First referring to FIG. 12, and with the applicator assembly 110 loaded onto the engagement surface 146, the user presses one end 120 of the applicator assembly first onto the selected test site, in this case, the upper arm. As the pair of applicators 114 shown on the far left side become firmly pressed against the patient skin, their low-durometer vial wall members 136 start to compress or flex, as described above in association with FIG. 10B, allowing respectively enclosed piercing elements (not shown) to extend and penetrate the skin, thereby injecting with their respective contents. The user then rolls the handle 142 in the direction shown by arrow 148. This rolling action causes the pressure point to move from the lower left end 120 of the applicator assembly 110 towards the upper right end 122 of the assembly, as new pairs of applicators start to inject into the skin in sequence while prior pairs detach from the skin. Note herein that a pin 150 fixedly couples the handle 142 to the rest of the actuator 140. In effect, the handle 142 and the body portion 144 are made integral through the pin 150, resulting in much assurance of the motion on the user's part. Alternatively, the handle 142 and the body portion 144 can be manufactured as a single integral piece. In another embodiment, and referring now to FIG. 13, the pin is removed, allowing the handle 142 to pivot about the actuator body portion 144. This provides an alternative pressing and rolling action.

Again, the application of multiple allergens through one common applicator assembly 110, especially when the application process is accomplished through an actuator 140 that has a singular engagement surface 146, uniformity in depth control is much more likely to be achieved. This form of application provides at least one major advantage over prior art applicators that require an operator to dip a piercing means into each well of allergen before individually applying the piercing means to the skin.

### PARTS LIST FOR FIGS. 1-13

20 disposable allergy-testing patch
22 patch body
23 position notch, patch
24 substrate
25 top surface, patch
26 packet
27 bottom surface, patch
28 marking material
29 central bore, patch
30 removable cover
32 adhesive
34 liquid allergen
36 freeze-dried allergen
37 separation barrier, packet
38 liquid solvent
40 actuator
42 compact body, actuator
44 engagement surface
46 channel
47 piercing element
48 central pin
50 button
52 arrow (direction of actuation)
54 protrusion
60 fluid retaining end
62 shoulder
70 all-in-one patch
71 support, piercing element
72 rim
74 rack
76 rack
78 cutout
80 actuator
90 scope
92 body, scope
94 distal end, scope
96 eyepiece
98 lens
100 engagement surface
102 hinge
104 tab
106 light switch
110 all-in-one applicator assembly
112 bridge
114 applicator
116 cutout
118 extension
120 one end along long axis, all-in-one applicator assembly
122 other end along long axis, all-in-one applicator assembly
124 vial
125 body, piercing element
126 base
127 tip end, piercing element
128 sealing strip
129 long axis, applicator
130 piercing tip
132 shoulder
134 distal rim, vial
136 vial wall member
138 flex point
140 rolling actuator
142 handle
144 body portion, actuator
146 engagement surface
148 arrow, rolling direction
150 pin, rolling actuator

While the present invention has been particularly shown and described with reference to the structures and methods disclosed herein and as illustrated in the drawings, it is not confined to the details set forth and this invention is intended to cover any modifications and changes as may come within the scope and spirit of the following claims.

1. A disposable allergy-testing patch, said patch comprising:
a flexible body having a plurality of packets, each housing an allergen and at least one piercing element, said at least one piercing element being movably supported within each said packet, and said piercing element including retaining means for retaining a portion of said allergen when at least a portion of said piercing element is forced through each said packet; and
at least one transferable marking material adjacent each said packet and disposed on a surface of said flexible body for marking a patient's body upon close contact therewith.

2. The patch of claim 1, wherein said patch further includes actuation means for moving said at least one piercing element at least partly out of each said packet to enable a portion of said piercing element, including said retaining means, to extend outwardly from said body.

3. The patch of claim 2, wherein each said actuation means comprises a tubular section made from an elastomeric material and is supported by at least a portion of said piercing element, each said tubular section being configured to deform upon compressive contact of said surface of said patch with the skin of a patient, thereby permitting said piercing element to move in relation to said tubular portion into direct contact with said skin.

4. The patch of claim 1, wherein said patch includes a removable portion covering said surface.

5. An allergy-testing apparatus comprising:
a disposable patch comprising a flexible body having at least one packet housing an allergen;
at least one transferable marking material adjacent said at least one packet and disposed on a surface of said flexible body for marking a patient's skin upon close contact therewith; and
at least one piercing element movably contained within said patch and configured to pierce a portion of said at least one packet, said at least one piercing element including retaining means for retaining a portion of said allergen, wherein said apparatus is configured to advance said retaining means towards said surface of said flexible body and said at least one transferable marking material into close contact with said patient's skin.

6. The apparatus of claim 5, including a removable strip covering said surface of said flexible body.

7. An allergy-testing apparatus comprising:
an actuating device comprising a compact body, an actuator mechanism and an engagement surface, said actuator mechanism being operatively connected to at least a portion of said engagement surface such that operation of said actuator mechanism causes spatial displacement of said at least a portion of said engagement surface relative to said compact body;
a packet assembly comprising at least one packet housing an allergen, said packet assembly being releasably attached to said engagement surface of said actuating device; and
at least one piercing element having a fluid retaining end, wherein spatial displacement of said engagement surface caused by said operation of said actuator mechanism deploys said piercing element such that said fluid retaining end pierces at least one side or said packet, enabling administration of said allergen to a patient.

8. The apparatus of claim 7, wherein said packet assembly further comprises a transferable marking material adjacent said at least one packet for marking a patient's skin upon close contact therewith.

9. The apparatus of claim 7, wherein said marking material comprises a material visible in ambient light.

10. The apparatus of claim 7, wherein said marking material comprises a pattern transferable to skin upon close contact therewith.

11. The apparatus of claim 7, wherein said packet assembly is flexible.

12. The apparatus of claim 7, wherein said packet assembly is disposable.

13. The apparatus of claim 7, wherein said body comprises an adhesive side coupled to a removable backing strip, said adhesive side enabling adherence to the skin of a patient.

14. The apparatus of claim 7, wherein said packet assembly comprises a plurality of packets and piercing elements, each of said packets housing a piercing element and at least one of a different allergen and a control material.

15. The apparatus of claim 14, wherein said actuator mechanism deploys all of said piercing elements at once.

16. The apparatus of claim 14, wherein said actuator mechanism deploys a portion of said piercing elements at a time.

17. An allergy-testing apparatus comprising:
a scope comprising a compact hand-held body, an illumination assembly disposed in connection with said body, an optical system disposed within said body, and an engagement surface; and
a substrate comprising at least one packet housing an allergen and a piercing element, said substrate being releasably attached to said engagement surface of said scope.

18. The apparatus of claim 17, wherein said substrate is disposable.

19. The apparatus of claim 17, wherein said scope includes an actuator mechanism for selectively deploying said piercing element and said allergen relative to a target in contact with said allergy-testing apparatus.

20. The apparatus of claim 17, wherein said optical system includes at least one lens and an eyepiece aligned along an optical axis, said optical axis extending through said engagement surface.

21. The apparatus of claim 17, wherein said substrate includes a plurality of packets, each of said packets housing a different allergen and a piercing element.

22. The apparatus of claim 17 wherein said scope further comprises a measuring reticle.

23. A disposable allergen-testing applicator comprising:
at least one piercing element having an elongated body extending from a base end to a tip end, said tip end comprising at least one sharp tip, said body comprising a shoulder at a predetermined distance relative to said sharp tip, said tip end containing an allergen suspended thereon by at least one capillary action and surface adhesion;
at least one enclosure into which said at least one piercing element is disposed, said enclosure having an opening that permits the passage of said tip end, said enclosure being made from a deformable material enabling the tip end of said piercing member to extend beyond said opening upon application of an external force onto said applicator; and
a sealing strip covering said enclosures and said piercing elements.

24. The applicator of claim 23, wherein each said enclosure is defined by a substantially Y-shaped wall member around the body of said piercing element, including said tip end, said wall member closely enclosing a portion of said piercing element from said base end of said piercing element to a flex point not beyond said shoulder of said piercing element, said wall member extending away from said body of said piercing member between said flex point and said tip end of said piercing element, said wall member terminating in a rim; and
a removable cover sealing said rim of said wall member and forming an interstice around said piercing element's tip end.

25. The applicator of claim 23, wherein said at least one enclosure is made from a low durometer elastomer.

26. An allergen-testing apparatus comprising:
a body comprising a curved engagement surface;
a handle coupled to said body such that operation of said handle causes movement of said curved engagement surface; and
a disposable applicator removably attached to and in conforming relationship with said curved engagement surface, said applicator comprising a plurality of piercing elements, each element having an allergen disposed on a tip end thereof, and a corresponding plurality of enclosures retaining said piercing elements, including associated tip ends, each of said enclosures being deformable based on a force applied by a user through said handle to permit the tip end of at least one enclosure to extend, thereby permitting administration of said allergen to the skin of a patient.

27. The apparatus of claim 26, wherein said engagement surface has a substantially convex configuration,

28. The apparatus of claim 26, wherein at least one of said body and said handle are reusable.

29. The apparatus of claim 26, wherein said body and said handle are integral.

30. The apparatus of claim 26, wherein operation of said handle causes sequential administration of allergens by said piercing elements to the skin of a patient.
